(19) United States
(12) Patent Application Publication
(10) Pub. No.: US 2015/0265331 A1
(43) Pub. Date: Sep. 24, 2015

(54) DEVICES FOR REDUCING LUNG VOLUME AND RELATED METHODS OF USE

(71) Applicant: Boston Scientific Scimed, Inc., Maple Grove, MN (US)

(72) Inventors: Sean P. FLEURY, Brighton, MA (US); Jason WEINER, Grafton, MA (US); Paul SMITH, Smithfield, RI (US); Robert B. DEVRIES, Northborough, MA (US); Gary J. LEANNA, Holden, MA (US); Man Minh NGUYEN, Harvard, MA (US)

(73) Assignee: Boston Scientific Scimed, Inc.

(21) Appl. No.: 14/660,200

(22) Filed: Mar. 17, 2015

Related U.S. Application Data
(60) Provisional application No. 61/954,694, filed on Mar. 18, 2014.

(51) Int. Cl. A61B 18/04

(52) U.S. Cl. CPC A61B 18/04; A61B 2018/00541

(57) ABSTRACT

A method for isolating a portion of a lung may include inserting a treatment device into an airway of a patient, and applying energy from the treatment device to a treatment site in the airway to at least partially occlude the airway to inhibit air from entering the airway distal to the treatment site.

### CROSS-REFERENCE TO RELATED APPLICATION

This patent application claims benefit of priority under 35 U.S.C. §119 to U.S. Provisional Patent Application No. 61/954,694, filed Mar. 18, 2014, the entirety of which is incorporated herein by reference.

### FIELD OF THE DISCLOSURE

This disclosure relates generally to devices for reducing lung volume and related methods of use. More particularly, the disclosure relates to methods and devices for delivering energy to an airway wall of a lung to reduce at least one symptom of a lung condition.

### BACKGROUND OF THE DISCLOSURE

Chronic obstructive pulmonary disease (COPD) is a progressive disease that affects breathing efficiency and lung capacity. COPD includes conditions such as chronic bronchitis and emphysema. COPD currently affects over 15 million people in the United States and is currently the third leading cause of death in the country. The primary cause of COPD is inhalation of cigarette smoke, responsible for over 90% of COPD cases. The economic and social burden of the disease is substantial and is increasing.

Emphysema is a long-term lung disease characterized by destruction of the lung tissue (e.g., lung parenchyma). The lung parenchyma is the tissue that supports the shape and function of the lungs. Thus, emphysema leads to loss of elastic recoil and tethering which maintains airway patency, reducing the ability of the lungs to exhale. Also, as bronchioles are not supported by cartilage like larger airways, they have little intrinsic support and therefore, are susceptible to collapse when destruction or tethering occurs, particularly during exhalation. The destruction of the lung tissue is mainly caused by destruction of structures feeding the alveoli. Also, in some cases it may be associated with deficiency of alpha 1-antitrypsin.

Smoking is one major cause of the destruction of the lung tissue that leads to the collapse of small airways in the lungs during forced exhalation. This leads to limited gas exchange and the trapping of air in the lungs. The trapping of air leads to increased concentrations of carbon dioxide in the blood which can cause shortness of breath (dyspnea) during physical activity, and an expanded chest.

Initially, when emphysema is mild, dyspnea occurs only during physical activity. When healthy, alveolar sacs are clustered like bunches of grapes. As emphysema worsens with time, the alveolar sacs transform into large and irregular pockets with holes in their inner walls. This in turn reduces the surface area of the lung tissue and limits gas exchange, reducing oxygen levels in the blood. Dyspnea can then occur even after little physical exertion. If the emphysema becomes sufficiently advanced, the victim may experience shortness of breath at all times, even during rest. Increased effort during breathing, the use of additional muscles during breathing, and blood gas abnormalities then combine to cause tachypnea, or rapid breathing that can continually worsen.

In some patients, acute exacerbations of COPD (AECOPD) may lead to worsening of symptoms, for example, an increase in or onset of cough, wheeze, and sputum changes. Various factors such as bacterial infection, viral infection, or pollutants, trigger AECOPD and lead to significant airway restriction.

Emphysema may be treated by various procedures such as lung volume reduction surgery (LVRS). LVRS involves resection and removal of damaged portions of the lungs to create more space in the thoracic cavity for healthy tissue to expand into. The removal of the damaged tissue allows the healthy portions of the lungs to function normally, enhancing breathing capability. LVRS is particularly effective for treating the upper lobes of the lungs. However, there are post-operative risks associated with LVRS such as blood loss, internal bleeding, and extensive damage to the lungs that may lead to death of the patient. As an alternative to LVRS, less invasive treatments including bronchial blocking devices (e.g., spigots or unidirectional valves), sealants, coils, vapor, and/or airway bypass systems are used to treat emphysema. However, these less invasive treatments may lead to the blockage of healthy portions of the lung among other complications, leading to further inefficient breathing.

Thus, there remains a need for improved methods and devices that allow for better treatment of COPD patients.

### SUMMARY OF THE DISCLOSURE

Embodiments of the present disclosure relate to methods of treating airways.

In accordance with an embodiment, the present disclosure is directed to a method for isolating a portion of a lung. The method may include inserting a treatment device into an airway of a patient, and applying energy from the treatment device to a treatment site in the airway to at least partially occlude the airway to inhibit air from entering the airway distal to the treatment site.

Various embodiments of the disclosure may include one or more of the following aspects: wherein the energy is applied to completely occlude the airway to isolate the portion of the lung that is distal to the airway from a remaining portion of the lung; deploying microparticles or nanoparticles into the airway; exciting the microparticles or nanoparticles to apply the thermal energy to the airway; wherein the treatment device is configured to deliver thermal energy via RF, microwave, ultrasound, light, or laser; deploying an electrode into the airway; wherein the electrode is formed on an expandable distal member; wherein the expandable distal member is a basket having a plurality of legs movable between a collapsed configuration and an expanded configuration, the plurality of legs being configured to contact a wall of the airway in the expanded configuration, the electrode being formed on at least one of the plurality of legs; deploying a balloon into the airway; wherein the balloon is inflated with a fluid to place an outer surface of the balloon in contact with the airway wall; wherein the fluid is heated above or below a temperature required to induce necrosis of cells in the airway to form scar tissue; wherein the balloon is a weeping balloon, and the method further includes applying a sclerosing agent through ports disposed on the outer surface of the balloon to create an inflammatory response in the airway; wherein occluding the airway further includes inserting a fluid delivery device through a wall of the airway; further including delivering an agent through the delivery lumen to induce necrosis of cells in the airway to form scar tissue; wherein the agent is ethanol or a spherical embolic; further including applying multiple energy modalities within the airway; wherein the portion of the lung includes emphysematous alveoli; further including removing air from the portion of the lung prior to applying the energy step.

In accordance with another embodiment, the present disclosure is directed to a method for isolating a portion of a lung. The method may include inserting a treatment device into an airway of a patient, and isolating the portion of the lung that is distal to the airway by applying a treatment via the treatment device to induce necrosis of cells in the airway.

In accordance with yet another embodiment, the present disclosure is directed to a method for isolating a portion of a lung. The method may include removing air from the portion of the lung, and inserting a treatment device into an airway of a patient. The method may further include, after removing air from the portion of the lung, applying energy from the treatment device to the airway to occlude the airway and isolate the portion of the lung that is distal to the airway.

Additional characteristics, features, and advantages of the disclosed subject matter will be set forth in part in the description that follows, and in part will be apparent from the description, or may be learned by practicing the disclosure. The characteristics and features of the disclosure can be realized and attained by way of the elements and combinations particularly pointed out in the appended claims.

It is to be understood that both the foregoing general description and the following detailed description are exemplary and explanatory only, and are not restrictive of the disclosed subject matter.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1 is an in vivo illustration of an exemplary medical device inserted into an airway of the lung, according to one embodiment of the present disclosure; and

FIGS. 2-7 illustrate side views of treatment devices according to various embodiments of the present disclosure.

### DESCRIPTION OF THE EMBODIMENTS

Reference will now be made to certain exemplary embodiments of the present disclosure, examples of which are illustrated in the accompanying drawings. Wherever possible, the same reference numbers will be used throughout the drawings to refer to the same or like parts. The term “distal” refers to the end farthest away from a medical professional when introducing a device in a patient. The term “proximal” refers to the end closest to the medical professional when placing a device in the patient.

### Exemplary Embodiments

The embodiments disclosed herein include methods and devices for treating a respiratory airway. However, it should be noted that the present disclosure contemplates use of the methods and devices for treatment of other body regions and/or tissue, such as renal nerves, bladder tissue, or the like. In addition to diagnosed airway diseases such as COPD, asthma, chronic cough, chronic bronchitis, and cystic fibrosis, other diseases such as bronchial hyperactivity associated with congestive heart failure and mitral valve stenosis may also be treated using the methods and devices disclosed in the present disclosure.

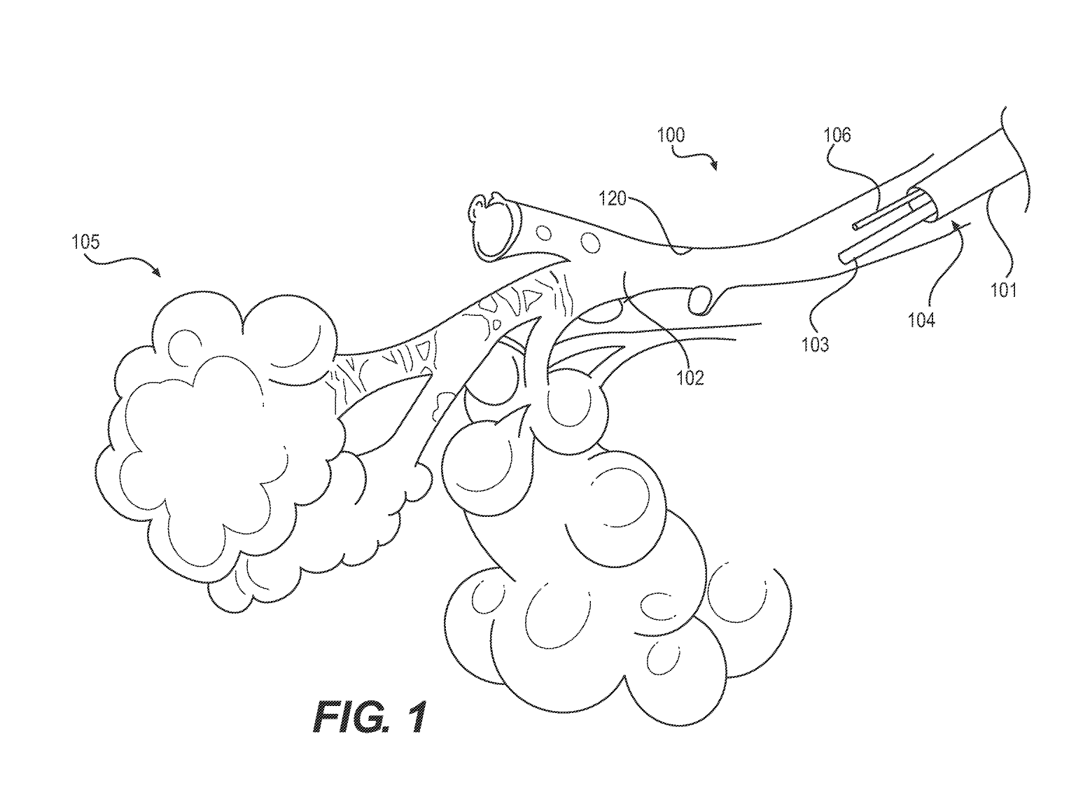

FIG. 1 shows a portion of a diseased lung including damaged tissue 105. In an exemplary embodiment, the damaged tissue 105 may be alveolar sacs and/or damaged airways that may exhibit symptoms caused by emphysema. In general, upon developing emphysema, the alveolar sacs may lose their elasticity and the ability to recoil during exhalation. Therefore, inhaled air may get trapped within the damaged tissue 105, causing a build-up of carbon dioxide in the damaged tissue 105. The damaged tissue 105 may be treated, removed, or isolated from the remaining (and healthy) portion of the lungs in order to prevent carbon dioxide build-up and associated complications. This may be achieved by occluding the airway at a treatment location that is proximal to damaged tissue 105.

In the illustrated embodiment, a medical device 100 may be inserted into a diseased airway 102. The medical device 100 may be configured to apply energy to a treatment location (e.g., airway 102 including, but not limited to terminal and/or non-terminal bronchioles, or other suitable airways). The medical device 100 may include an elongate member 101, and a treatment device 103 extending from a distal end 104 of the elongate member 101.

In some embodiments, the medical device 100 may be a bronchoscope, a catheter shaft, or another suitable elongate member. Elongate member 101 may include one or more lumens extending longitudinally along the length of the elongate member 101. In some embodiments, the elongate member 101 may include an actuation mechanism (not shown) such as a handle and push-pull member configured to move the treatment device 103 from an undeployed position within elongate member 101 to a deployed position distal to elongate member 101. It is further contemplated that other suitable actuation mechanisms alternatively may be utilized.

The elongate member 101 may be formed of any suitable material. Examples of such materials may include, but are not limited to, silicone, polyurethane, PVC or the like. In some embodiments, these materials may exhibit sufficient flexibility to be maneuvered through and positioned within airway 102 without causing any injury to the surrounding tissue, such as, e.g., healthy airway walls 120. In some embodiments, these materials may include internal and/or external layers of lubricious materials in order to facilitate easy insertion of the medical device 100 into the airway.

Prior to the introduction of the medical device 100, air from the damaged tissue 105 may be removed using a suction device 106 so that air does not become trapped within damaged tissue 105 after the airway 120 is occluded. Suction device 106 may be a catheter or other suitable member coupled at a proximal end to a negative pressure source, e.g., a pump. In some embodiments, the treatment device 103 may be deployed into airway 102 after air is removed from damaged tissue 105 by suction device 106.

The treatment device 103 may be any suitable treatment device configured to at least partially occlude airway 102 to isolate damaged tissue 105 from a remaining portion of the lung to inhibit air from entering airway 102 distal to a treatment site. In some embodiments, energy applied by treatment device 103 may completely occlude the airway 102 at the treatment site to isolate damaged tissue 105 from a remaining portion of the lung. In some embodiments, the treatment device 103 may be used to deliver thermal energy at the treatment location. In other embodiments, any other suitable form of energy such as, e.g., mechanical, chemical, radio frequency, radioactive, ultrasonic, light, or the like, may be utilized for the treatment. In some embodiments, one or more energy modalities may be applied to occlude the airway 102.

In some embodiments, the application of energy may induce necrosis of cells in airway 102 to form scar tissue sufficient to occlude airway 102. In some embodiments, the treatment location may be located proximal to the damaged tissue 105 in an airway 102 that is upstream of the damaged tissue 105. In certain other embodiments, the treatment location may be chosen based on ease of access or to minimize damage to healthy tissue. In some embodiments, the treatment may be applied to multiple airways 102 of the lung. In some embodiments, the treatment may be applied to multiple locations along the same airway 102.

Alternatively or additionally, all or a portion of medical device 100, elongate member 101, and/or treatment device 103 may be formed of a radiopaque material so that it can be visualized under fluoroscopic guidance, or may otherwise include radiopaque or other imaging markers for guidance. The markers may be used to ensure that a correct direction of therapy is applied. In some embodiments, treatment device 103 may be prevented from activating until the marker is appropriately positioned.

Additionally, the medical device 100 may include one or more temperature sensors to measure the temperature of airway 102 during therapy. Feedback mechanisms (e.g., PID loops) may be employed to control the temperature of the airway 102 to induce necrosis of the cells in the airway. Necrosis may be premature cell death induced by medical device 100, and may include cell membrane disruption, ATP depletion, metabolic collapse, cell swelling, cell rupture, and inflammation. In some embodiments, sensing devices may be employed to detect structures within the airway such as blood vessels that are to be preserved. In some embodiments, Doppler ultrasound sensors, imaging systems, or the like may be utilized. In some embodiments, an efficacy of the treatment may be determined by measuring electrical signals of nerve traffic, radial force in airway, or by other measurements.

Medical device 100 may carry out the methods described herein utilizing one or more devices or features disclosed in U.S. Pat. No. 7,425,212, issued on Sep. 16, 2008, U.S. Pat. No. 6,488,673, issued on Dec. 3, 2002, U.S. Pat. No. 8,257,413, issued on Sep. 4, 2012, and U.S. Patent Application Publication No. 2014/0018789, published on Jan. 16, 2014, the entireties of each of which are incorporated by reference herein.

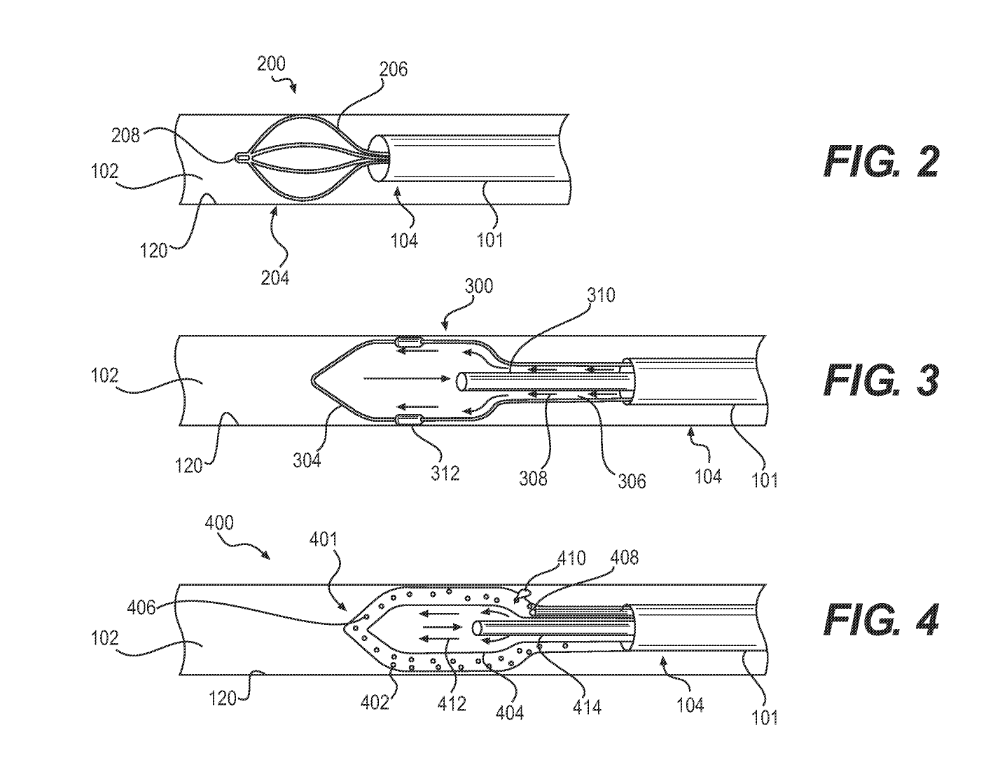

FIG. 2 illustrates an exemplary treatment device 200 extending from distal end 104 of elongate member 101. The treatment device 200 may include an expandable distal member such as an expandable basket 204 configured to be reciprocally movable between an expanded configuration (shown in FIG. 2) and a collapsed/retracted configuration (not shown). In some embodiments, expandable basket 204 may instead be arranged as a nest, globe, or other suitable expandable member.

In some embodiments, expandable basket 204 may include a plurality of legs 206 through which the thermal energy may be applied. In some embodiments, the legs 206 may be coupled at a distal tip 208 using any suitable technique such as, but not limited to soldering, welding, or the like. However, in other embodiments, the legs 206 may not be connected at the distal tip 208, and the expandable distal member may be formed as a prong or another suitable shape. In some embodiments, an arcuate surface of the legs 206 may come in contact with the airway wall 120 when the expandable basket 204 is expanded radially. The shape and size of the expandable basket 204 can be adjusted to create larger or more localized burn areas depending on the application. The legs 206 may be partially coated with an insulating material except for arcuate surfaces that may transfer thermal energy to or from the airway wall 120.

In the expanded configuration, the legs 206 may be in close proximity to or in contact with the airway wall 120 to apply energy to the airway wall 120. The applied energy may be at a sufficient temperature and persist for a sufficient time period to induce necrosis, but not apoptosis, of cells in airway 102. Necrosis may lead to sufficient scar tissue formation in airway 102 to occlude the airway 102, isolating damaged tissue 105 (referring to FIG. 1) distal to the applied treatment areas. Thus, after applying the treatment to airway 102, air flow may be impeded to the damaged tissue 105. In some embodiments, cells in airway 102 may be heated to a temperature of about 50° C. to 110° C., although other suitable temperatures are also contemplated.

Expandable basket 204 may be formed of any suitable material including biocompatible metals, alloys, or other materials. In some embodiments, expandable basket 204 may be formed from stainless steel, aluminum, or the like. In some embodiments, at least some portions of expandable basket 204 may include an insulating coating such as but not limited to PVC, Teflon, silicon, or the like.

A treatment device 300 is shown in FIG. 3. In some embodiments, the treatment device 300 may include an expandable distal member such as a balloon 304 extending from the distal end 104 of the elongate member 101. A circulating fluid 308 may be delivered to balloon 304 through a lumen 306 to inflate the balloon 304. Once inflated, an outer surface of the inflatable balloon 304 may contact airway wall 120. As shown, balloon 304 may include one or more energy delivery devices 312 disposed along the outer surface of the inflatable balloon 304. In some embodiments, energy delivery devices 312 may be electrodes configured to deliver thermal energy to airway wall 120. The energy delivery devices 312 may be configured to generate sufficient localized thermal energy to ablate portions of airway 102 to create scar tissue occluding airway 102.

In some embodiments, the energy delivery devices 312 may be configured to deliver RF, microwave, laser, or another suitable energy modality. In some embodiments, one or more energy modalities may be applied by energy delivery devices 312. Energy delivery devices 312 may be supplied with energy from a console unit (not shown) through the wires, conductors, or other suitable members traversing through or around the elongate member 101.

In some embodiments, fluid 308 may be utilized to provide a treatment. In such embodiments, the fluid 308 may be heated to a temperature sufficient to create scar tissue in airway 102 by necrosis. In some embodiments, the fluid 308 may be heated locally within balloon 304 via heating elements electrically coupled to an energy source (not shown). In other embodiments, the fluid 308 may be heated prior to its delivery to balloon 304.

In some embodiments, the fluid 308 may be a cryo-fluid such as liquid nitrogen, or another cooled fluid. Thus, in some embodiments, fluid 308 may be cooled to a temperature sufficient to create scar tissue in airway 102 by necrosis of cells in airway 102. In other embodiments, fluid 308 may be a cooled fluid that is circulated through balloon 304 while energy delivery device 312 applies thermal energy to airway 102, preventing excessive damage to healthy tissues.

During or after energy delivery to the airway 102, fluid 308 may be circulated through balloon 304. Lumen 306 and conduit 310 may ensure circulation of fluid 308 through balloon 304. Balloon 304 may be formed from a flexible material that can be inflated and/or may be capable of transferring heat to or from airway 102. In some embodiments, treatment device 300 may not include energy delivery devices 312.

A treatment device 400 is depicted in FIG. 4. The treatment device 400 may include an expandable distal member 401 extending distally from the elongate member 101. The expandable distal member 401 may include an inner inflatable member 404, and an outer inflatable member 402 disposed around the inner inflatable member 404. In some embodiments, the inner inflatable member 404 may be a sealed member configured to receive a fluid 412. Inner inflatable member 404 and fluid 412 may be substantially similar to balloon 304 and fluid 308 described with reference to FIG. 3. The fluid 412 may inflate the inner inflatable member 404 such that the outer inflatable member 402 may come in contact with the airway wall 120. Fluid 412 may be heated or cooled in a substantially similar manner as fluid 308 described with reference to FIG. 3. The outer inflatable member 402 may be inflated with a fluid 410 to come into contact with and/or deliver fluid 410 to the airway wall 120. The fluid 410 may be delivered through a conduit 408 that extends from a proximal end (not shown) of elongate member 101. Thus, outer inflatable member 402 may be a balloon with weeping capabilities (a weeping balloon). The outer inflatable member 402 may have openings 406 through which the fluid 410 may flow. The fluid 410 may be heated, cooled, or otherwise configured to create necrosis. The fluid 410 may directly contact airway wall 120 leading to scar tissue formation. In some embodiments, fluid 410 may be a sclerosing agent that induces an inflammatory response in airway 102, increasing scar tissue formation. In some embodiments, the sclerosing agent may include one or more of polidocanol, ethanolamine oleate, morrhuate sodium, sodium tetradecyl sulfate, or other suitable sclerosing agents.

The inner inflatable member 404 and the outer inflatable member 402 may be formed of any suitable material. In some embodiments, inner inflatable member 404 and outer inflatable member 402 may be formed of a continuous, e.g., monolithically formed unitary structure. In some embodiments, the inner inflatable member 404 and the outer inflatable member 402 may be discrete components that are later coupled together. The inner inflatable member 404 and the outer inflatable member 402 may be formed of same or different material such as, e.g., silicone, PVC, Polyurethane, or the like.

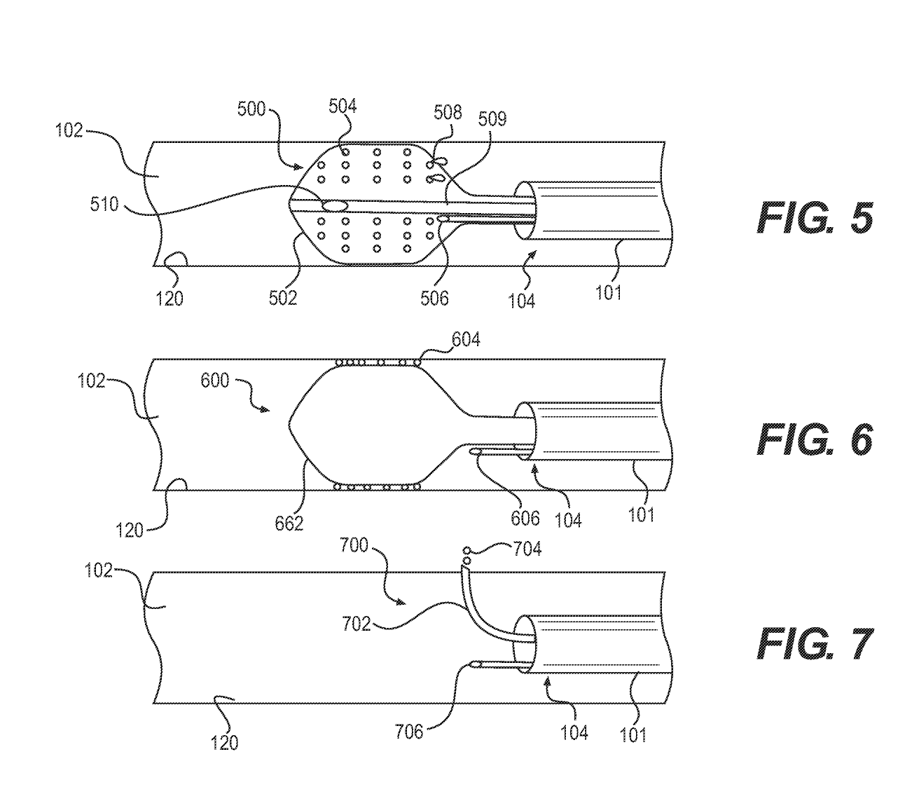

A treatment device 500 is depicted in FIG. 5. The treatment device 500 may include an elongate member 509, and an expandable distal member such as a balloon 502 disposed over the elongate member 509. The balloon 502 may have weeping capabilities and may be substantially similar to outer inflatable member 402 described with reference to FIG. 4. The elongate member 509 and the inflatable balloon 502 may extend distally from elongate member 101. An energy delivery device 510 may be disposed along the elongate member 509 to deliver energy to the airway wall 120. In some embodiments the energy delivery device 510 may be a radio frequency (RF) electrode configured to deliver RF energy to airway 102. The RF energy may be delivered partially or completely along the circumference of the airway 102. In some embodiments, the energy delivery device 510 may be configured to deliver microwave energy, ultrasound energy, or another suitable energy modality. The energy delivered via energy delivery device 510 may induce necrosis of cells in airway 102 to occlude airway 102.

Balloon 502 may include a plurality of openings 504 defined along an outer surface of balloon 502. The openings 504 may be configured to deliver fluid 508, such as, e.g., a gel, a heated fluid, a cooled fluid, a sclerosing agent, or another substance to airway 102 before, during, or after energy is delivered via energy delivery device 510. When fluid 508 is a sclerosing agent, it may induce an inflammatory response in the airway 102 increasing scar tissue formation. In some embodiments, the treatment device 500 may include a conduit 506 configured to deliver the fluid 508 to balloon 502.

A treatment device 600 is depicted in FIG. 6. The treatment device 600 may include an expandable distal member such as a balloon 602 extending distally from elongate member 101. The balloon 602 may be substantially similar to balloon 304 described with reference to FIG. 3. The treatment device 600 may further include particles 604 such as, e.g., nanoparticles, microparticles, or other suitable particles, that can be placed on an outer surface of the balloon 602. When balloon 602 is inflated, the particles 604 may be delivered to the surface of airway wall 120. In some embodiments, particles 604 may be embedded beyond the surface of airway wall 120 into the lung parenchyma. In some embodiments, the particles 604 may bind to the airway wall 120 using any suitable bio-molecular linker. Once attached or embedded, the particles 604 may be activated to generate thermal energy sufficient to destroy the tissue of airway 102, inducing scar tissue formation. An intensity of the thermal energy generated by the particles 604 may vary based upon the number of particles 604 deployed to airway wall 120, among other factors.

Particles 604 may be metallic, organic, inorganic, water-based, gel-based, colloidal, or another suitable type of particles and combinations thereof. In some embodiments, an energy delivery device 606 configured to activate particles 604 may extend through the elongate member 101. In some embodiments, particles 604 may be optically activated to generate thermal energy such that the particles 604 may absorb photons to generate thermal energy. For optically activating the particles 604, energy delivery device 606 may be a light source emitting light in any suitable wavelength (e.g., visible, UV, or infrared). In some embodiments, particles 604 may be activated by RF, ultrasound, or another suitable mechanism. It is also contemplated that energy delivery device 606 may be deployed to airway 102 by another suitable mechanism. In some embodiments, energy delivery device 606 may be located outside of the body. In some embodiments, particles 604 may be radiopaque, fluorescent, or be otherwise detectable within the airway 102.

A treatment device 700 is depicted in FIG. 7. In some embodiments, the treatment device 700 may include a fluid delivery device 702, such as, e.g., an injection needle or syringe that may extend distally from distal end 104 of elongate member 101 at the treatment location. The fluid delivery device 702 may be configured for injecting particles 704 such as, e.g., nanoparticles, heating fluids, cooling fluids, or other substances into airway 102 or through airway wall 120. Particles 704 may be activated by an energy delivery device 706. Particles 704 and energy device 706 may be substantially similar to particles 604 and energy delivery device 606 described with reference to FIG. 6.

In some embodiments, particles 704 may be a fluid configured to induce scar tissue formation in airway 100. In some embodiments particles 704 may be ethanol, a contoured spherical embolic, or another suitable substance configured to cause necrosis of cells in airway 102. In some embodiments, the fluid delivery device 702 may have a beveled distal end to facilitate piercing tissue. In some embodiments, the fluid delivery device 702 may be pre-bent radially outward from a longitudinal axis of elongate member 101. That is, in a first configuration, fluid delivery device 702 may be constrained within elongate member 101. While in the first configuration, fluid delivery device 702 may be displaced distally from distal end 104 of elongate member 101 into a second configuration. Fluid delivery device 702 may expand radially outward in the second configuration and pierce through airway wall 120 as shown in FIG. 7. Any suitable number of fluid delivery devices 702 may be displaced from distal end 104 of elongate member 101. The fluid delivery device 702 may be made from any suitable material such as but not limited to stainless steel, aluminum, titanium, or the like.

Embodiments of the present disclosure may be used in many different medical or non-medical environments. In addition, at least certain aspects of the aforementioned embodiments may be combined with other aspects of the embodiments, or removed, without departing from the scope of the disclosure.

Other embodiments of the present disclosure will be apparent to those skilled in the art from consideration of the specification and practice of the embodiments disclosed herein. It is intended that the specification and examples be considered as exemplary only, with a true scope and spirit of the disclosure being indicated by the following claims.

1. A method for isolating a portion of a lung, the method comprising:
inserting a treatment device into an airway of a patient; and
applying energy from the treatment device to a treatment site in the airway to at least partially occlude the airway to inhibit air from entering the airway distal to the treatment site.

2. The method of claim 1, wherein the energy is applied to completely occlude the airway to isolate the portion of the lung that is distal to the airway from a remaining portion of the lung.

3. The method of claim 1, further including deploying microparticles or nanoparticles into the airway.

4. The method of claim 3, further including exciting the microparticles or nanoparticles to apply the thermal energy to the airway.

5. The method of claim 1, wherein the treatment device is configured to deliver thermal energy via RF, microwave, ultrasound, light, or laser.

6. The method of claim 1, further including deploying an electrode into the airway.

7. The method of claim 6, wherein the electrode is formed on an expandable distal member.

8. The method of claim 7, wherein the expandable distal member is a basket having a plurality of legs movable between a collapsed configuration and an expanded configuration, the plurality of legs being configured to contact a wall of the airway in the expanded configuration, the electrode being formed on at least one of the plurality of legs.

9. The method of claim 1, further including deploying a balloon into the airway.

10. The method of claim 9, wherein the balloon is inflated with a fluid to place an outer surface of the balloon in contact with the airway wall.

11. The method of claim 9, wherein the fluid is heated above or below a temperature required to induce necrosis of cells in the airway to form scar tissue.

12. The method of claim 9, wherein:
the balloon is a weeping balloon; and
the method further includes applying a sclerosing agent through ports disposed on the outer surface of the balloon to create an inflammatory response in the airway.

13. The method of claim 1, wherein occluding the airway further includes inserting a fluid delivery device through a wall of the airway.

14. The method of claim 13, further including delivering an agent through the delivery lumen to induce necrosis of cells in the airway to form scar tissue.

15. The method of claim 14, wherein the agent is ethanol or a spherical embolic.

16. The method of claim 1, further including applying multiple energy modalities within the airway.

17. The method of claim 1, wherein the portion of the lung includes emphysematous alveoli.

18. The method of claim 1, further including removing air from the portion of the lung prior to applying the energy step.

19. A method for isolating a portion of a lung, the method comprising:
inserting a treatment device into an airway of a patient; and
isolating the portion of the lung that is distal to the airway by applying a treatment via the treatment device to induce necrosis of cells in the airway.

20. A method for isolating a portion of a lung, the method comprising:
removing air from the portion of the lung;
inserting a treatment device into an airway of a patient; and
after removing air from the portion of the lung, applying energy from the treatment device to the airway to occlude the airway and isolate the portion of the lung that is distal to the airway.
